(19) United States
(12) Patent Application Publication
(10) Pub. No.: US 2025/0000669 A1
(43) Pub. Date: Jan. 2, 2025

(54) THREE-DIMENSIONAL FUNCTIONAL IMPINGEMENT ANALYSIS IN TOTAL HIP ARTHROPLASTY

(71) Applicant: New York Society for the Relief of the Ruptured and Crippled, maintaining the Hospital for Special, New York, NY (US)

(72) Inventors: Jonathan M. Vigdorchik, New York, NY (US); Fernando J. Quevedo Gonzalez, Greenwich, CT (US); Eytan M. Debbi, New York, NY (US); Joseph D. Lipman, New York, NY (US); Timothy M. Wright, New York, NY (US)

(21) Appl. No.: 18/698,894

(22) PCT Filed: Oct. 7, 2022
(86) PCT No.: PCT/US22/46098

§ 371 Date: Apr. 5, 2024

Related U.S. Application Data
(60) Provisional application No. 63/253,255, filed on Oct. 7, 2021.

(51) Int. Cl. A61F 2/46; A61B 34/10; G06F 30/20

(52) U.S. Cl. CPC A61F 2/46; A61B 34/10; G06F 30/20; A61B 2034/104; A61B 2034/108; A61F 2002/4633

(57) ABSTRACT

In one or more implementations of the present disclosure, a computer-implemented system and method are provided for virtually optimizing implant configurations to maximize range of motion in connection with total hip arthroplasty. At least one computing device receives preoperative clinical data of a patient and generates three-dimensional geometries of the patient's bones using the preoperative clinical data. Moreover, the at least one computing device selects implant geometry associated with at least one implant. Using the geometries of the bones and the selected at least one implant, the at least one computing device determines a virtual implantation including a plurality of components. The at least one computing device defines bounds in connection with the virtual implantation, including for at least one of component selection, position, orientation, and offset.

### CROSS-REFERENCE TO RELATED APPLICATIONS

The present application is based on and claims priority to U.S. Provisional Patent Application Ser. No. 63/253,255, filed on Oct. 7, 2021, and entitled THREE-DIMENSIONAL FUNCTIONAL IMPINGEMENT IN TOTAL HIP ARTHROPLASTY: A BIOMECHANICAL ANALYSIS, the entire contents of which are hereby incorporated by reference as if expressly set forth in its entirety herein.

### FIELD

The present disclosure relates, generally, to data management and communications and, more particularly, to a system and method for a computerized simulation methodology to quantify the effect of arthroplasty component choice on the risk of impingement during functional positions, accounting for the effect of implant selection, position, orientation, and offset and bony orientation.

### BACKGROUND

Dislocation is a common complication following surgical procedures involving total hip arthroplasty (THA) or revision THA, and is a predominant cause of revision and re-revision THA surgery. Impingement, which frequently precedes dislocation, can be characterized as prosthetic-prosthetic, prosthetic-bone, and bone-bone. Prosthetic-prosthetic impingement following THA, for example, can involve the neck of the femoral component with the liner of the acetabular component. Prosthetic-bone impingement following THA, for example, can involve the neck of the femoral component with the acetabular bone rim. Furthermore, bone-bone impingement following THA can involve the lesser trochanter of the femur with the ischial tuberosity.

Prosthetic impingement is a frequently reported type of impingement, and is influenced by the position of the components. More particularly, prosthetic impingement can be affected by the orientation of the acetabular component, the offset and orientation of the femoral component, the choice of the femoral head, and the choice of the liner design (e.g., lipped liners or dual mobility articulations).

Bone-bone impingement, although less reported, is also a frequent source of impingement. Generally, bone-bone impingement can be affected by the distance between the femur and the pelvis. Such distance between the femur and pelvis can be expressed in terms of three distances or offsets: the acetabular offset; the femoral offset; and the combined offset thereof. More particularly, the acetabular offset is the distance between the center of the pelvis and the center of rotation of the hip. The femoral offset is the distance between the center of rotation of the hip and the long axis of the femur. The combined offset is the addition of the acetabular and femoral offsets.

The femoral offset and acetabular offset are commonly described and measured on two-dimensional anterior-posterior (AP) radiographs. These offsets are three-dimensional in nature, however, and are affected by the three-dimensional position of the components, the pelvis, and the femur. Unfortunately, patients often dislocate in positions that differ from positions described on an AP radiograph. For example, flexion and internal rotation of the hip, or extension and external rotation of the hip can result in dislocation. At these functional positions for dislocation, the combination of component orientation and the position of the femur relative to the pelvis can affect the value of the acetabular and femoral offsets.

Furthermore, while changes to the femoral offset affect the distance between the femur and the pelvis, exclusively, changes to the acetabular offset can also affect the center of rotation of the femur with respect to the pelvis. As a result, changes to the acetabular offset can impact the path of the femur during various three-dimensional movements. Consequently, the implications for impingement of changes to the offsets can depend on the respective position of the hip and the nature of the offset (i.e., femoral or acetabular).

It is recognized that femoral and acetabular offsets are influenced by the three-dimensional position and orientation of the femoral and acetabular components. As such, the choice and orientation of the components can affect the femoral and acetabular offset measurements. For example, the medialization or lateralization of the acetabular cup can result in a unidirectional change of the acetabular offset. However, a change in the liner offset can create a change in three directions, which depends on the orientation of the cup (i.e., anteversion and inclination) and, thus, have a different impact on the acetabular offset. Further, changes to the offset of the femoral component or the femoral head can lead to changes of the femoral offset in two or three directions. While these changes are dependent upon the orientation of the femoral component (e.g., femoral anteversion), their directions are different. Accordingly, they have different impacts on the femoral offset.

Further, medialization or lateralization of the acetabular cup or changes to liner offset both affect the center of rotation of the hip, while changes to the femoral head offset and femoral component offset do not impact the center of rotation of the hip. Changes to the center of rotation to the hip, in addition to altering offset, also impact the path of motion of the femur, further impacting impingement. Changes to the acetabular cup offset medialize or lateralize the center of rotation. Conversely, changes to the liner offset affect the center of rotation in three directions (anterior-posterior, medial-lateral, and proximal-distal), depending on the orientation of the acetabular cup (i.e., anteversion and inclination).

Determining how changes in offset affect range of motion leading to impingement and/or the location of impingement is complex. Femoral and acetabular offset changes are commonly described on two-dimensional anterior-posterior (AP) pelvis radiographs and can appear similar in direction. Patients, however, do not usually dislocate with the hip positioned as it is on an AP pelvic radiograph. Instead, patients usually impinge and dislocate their hip in impingement-prone functional positions which occur with sitting (flexion and internal rotation of the hip), as well as standing (extension and external rotation of the hip). In these positions, acetabular and femoral offset changes are complex and entail very different multi-dimensional vectors which may result in very different effects of each offset on bone-bone impingement.

Unfortunately, evaluating impingement and, accordingly, the likelihood of dislocation has been difficult to determine. This is at least in part due to an inability to quantify the three-dimensional effect of various offset changes thereto. While changes to offset are often reported as two-dimensional measures on AP pelvic radiographs, the position of the bones in dislocation-prone positions can be different from their position on AP radiographs. Furthermore, changes to offset often occur in three directions. Consequently, the two-dimensional measurements on AP radiographs may not adequately capture the magnitude of offsets or their effect on the relative positions of the bones in functional positions. Accordingly, recognizing and understanding the impact of component offset has been at least unclear.

The present system and method address these and other deficiencies in the art, and it is with respect to these and other considerations that the disclosure made herein is presented.

### BRIEF SUMMARY

In one or more implementations of the present disclosure, a computer-implemented system and method are provided for virtually optimizing implant configurations to maximize range of motion in connection with total hip arthroplasty. At least one computing device receives preoperative clinical data of a patient and generates three-dimensional geometries of the patient's bones using the preoperative clinical data. Moreover, the at least one computing device selects implant geometry associated with at least one implant. Using the geometries of the bones and the selected at least one implant, the at least one computing device determines a virtual implantation including a plurality of components. The at least one computing device defines bounds in connection with the virtual implantation, including for at least one of component selection, position, orientation, and offset. Thereafter, a change to an aspect of the virtual implantation in accordance with the bounds is applied by the at least one computing device and, using at least some of the preoperative clinical data, pelvic mobility is determined. In addition, the at least one computing device uses the applied change to the aspect of the virtual implantation and the determined pelvic mobility to determine impingement resulting from at least one position of the patient's hip. After determining the impingement, the at least one computing device evaluates at least one choice associated with the at least one implant configuration. Where the step of evaluating does not represent the choices are optimized, the at least one computing device applies a different change to an aspect of the virtual implantation, in accordance with the bounds, and determines impingement resulting from at least one position of the patient's hip and evaluates, after determining the impingement, at least one choice associated with an aspect of the virtual implantation. Where the step of evaluating does represent the choices are optimized, the at least one computing device outputs information representing maximized offset.

In one or more implementations of the present disclosure, the preoperative data includes at least one of a computed tomography scan, a magnetic resonance image, an x-ray, dynamic image output, and sensor output.

In one or more implementations of the present disclosure, the three-dimensional geometries of the bones are generated by segmentation.

In one or more implementations of the present disclosure, selecting the implant geometry includes: identifying, by the at least one computing device, a component identifier; and matching, by the at least one computing device, information stored in at least one database representing the implant geometry.

In one or more implementations of the present disclosure, the virtual implantation is determined using at least one of an anatomical-based position according to established clinical protocols and a user selection.

In one or more implementations of the present disclosure, defining the bounds is based on at least one of received information, information retrieved from a database, and information calculated using characteristics of a patient and an implant.

In one or more implementations of the present disclosure, the at least one functional position includes at least one of flexion, extension, abduction, adduction, internal rotation, and external rotation.

In one or more implementations of the present disclosure, the virtual implantation includes at least one of component selection, component position, component orientation, and component offset.

In one or more implementations of the present disclosure, the choices include component selection, position, orientation, and offset.

Additional features, advantages, and embodiments of the disclosure may be set forth or apparent from consideration of the detailed description and drawings. It is to be understood that the foregoing summary of the disclosure and the following detailed description and drawings provide non-limiting examples that are intended to provide further explanation without limiting the scope of the disclosure as claimed.

### BRIEF DESCRIPTION OF THE DRAWINGS

Aspects of the present disclosure will be more readily appreciated upon review of the detailed description of its various embodiments, described below, when taken in conjunction with the accompanying drawings, of which:

FIG. 1 is a block diagram illustrating an example implementation of the present disclosure and that represents a plurality of devices and the flow of information associated with the devices;

FIG. 2 is a block diagram that illustrates functional elements of one or more of a data processing apparatus or computing device;

FIG. 3 illustrates a computational workflow, in accordance with an example implementation of the present disclosure;

FIG. 4 illustrates an anatomical rendering and illustrating use of reference anatomical landmarks on the pelvis, in accordance with an example implementation of the present disclosure;

FIGS. 5A and 5B are anatomical renderings illustrating acetabular cup offset and liner offset, respectively, in accordance with an example implementation of the present disclosure;

FIGS. 6A and 6B are anatomical renderings illustrating femoral stem offset and femoral head offset, respectively, in accordance with an example implementation of the present disclosure; and

FIG. 7 illustrates a computational workflow, in accordance with an example implementation of the present disclosure involving computer modeling.

### DETAILED DESCRIPTION OF CERTAIN IMPLEMENTATIONS

By way of introduction and overview, the present disclosure includes systems and methods that operate to identify and provide information representing the effect of surgical choices in component position during THA on the range of motion to impingement. For example, features of the present disclosure quantify effects of acetabular and femoral offset and provides information representing their contribution to the risk of impingement in functional position following THA. More particularly, the present disclosure includes computerized simulation and quantifies the effect that THA component offset has on the risk of impingement during functional positions, including by accounting for the effect of implant orientation and bony orientation.

In one or more implementations, pre-operative computerized tomography (CT) scans, magnetic resonance imaging (MRI), or any other suitable three-dimensional technology capable of imaging the skeleton of a patient undergoing THA are utilized to obtain patient-specific geometries of the pelvis and femur. Using the three-dimensional images or the reconstructions of the pelvis, reference anatomical landmarks on the pelvis can be determined, including both anterior-superior iliac spines (ASIS), the pubic symphysis, and the center of acetabula. These are usable to define the three-dimensional orientation of the pelvis and provide references for orientation of the THA components. Particularly, the anterior-pelvic plane can be used to determine the frontal plane of the patient, while the medial-lateral direction can be determined from the line connecting both ASIS or the line connecting the center of the acetabula. It should be noted that a CT-scan of a patient with a previous contralateral total or partial hip replacement can also be used, in which case the center of the replaced hip is utilized instead of the center of the acetabula.

In addition to the pelvis, reference anatomical landmarks can be determined on the femur from the three-dimensional images or the reconstructed patient-specific geometry of the femur, including the center of the femoral head, the center of the knee, and the most posterior points on the femoral condyles. It should be noted that these landmarks, particularly those related to the knee, can be obtained on the native bony geometry of the patient or on any total or partial joint replacement implant. These landmarks can be used to define the three-dimensional orientation of the femur, as well as to provide references for orientation of the femur. For example, superior-inferior direction can be determined from the mechanical axis of the femur, which connects the center of the femoral head and the center of the knee, while medial-lateral direction can be determined from the posterior condylar axis, which connects the most posterior points of both femoral condyles.

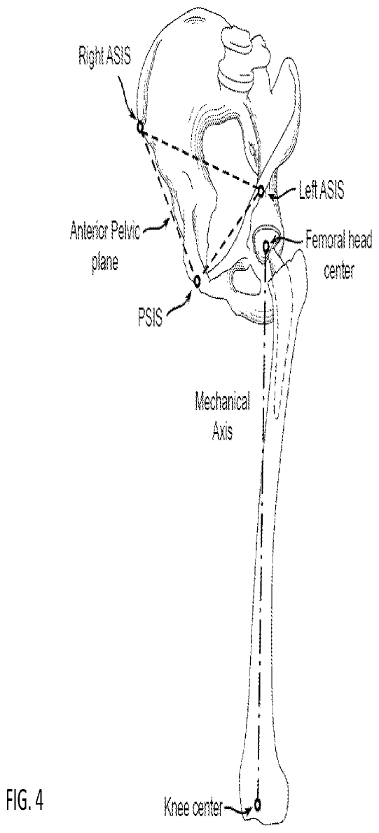

Using reference anatomical landmarks determined using one or more three dimensional images or their derived reconstructions of the pelvis and femur, the pelvis can be aligned to a reference orientation. For example, and with reference to FIG. 4, the anterior pelvic plane can be made coincident with the frontal plane and the medial-lateral axis, which can be defined either by a line connecting the center of both acetabula, a line connecting both ASIS, or other suitable definition, can be horizontal (i.e., parallel to the ground). One of ordinary skill will recognize that other suitable reference landmarks and orientations can be determined and used without departing from the teachings herein.

In addition, the femur can be aligned to a reference orientation relative to the pelvis, such that the medial-lateral axis of the femur can be parallel to the medial-lateral axis of the pelvis, and that the axis resulting from the cross-product of the mechanical axis of the femur and the medial-lateral axis of the femur can be aligned with the normal vector of the anterior pelvic plane. One of ordinary skill will recognize that the medial lateral axis of the femur can be defined by the posterior condylar axis or other suitable way. It should be noted that other suitable reference landmarks and orientations of the femur relative to the pelvis are possible.

In accordance with one or more implementations of the present disclosure, the pelvis and femur can be virtually implanted using the known three-dimensional geometries of the implants chosen for THA. For example, known geometries of the femoral component, the femoral head, the acetabular liner, and the acetabular cup are usable for virtually implanting the pelvis and femur. The initial implantation can follow standard guidelines for virtually positioning and orienting the components. For example, the acetabular cup may be placed to recreate the native center of rotation, with 40°±10° inclination and 15°±10° anteversion. As a result of the virtual implantation, the position of the femur relative to the pelvis may change. For example, femur may be distalized or lateralized. It should be noted that the initial position can also be decided by the surgeon on a patient-specific basis.

Moreover, one or more processors executing instructions can be configured to parametrically changing the position of the femoral and acetabular components independently. Changes in position to the acetabular cup, for example, can imply changes in the medial-lateral direction (i.e., changes in offset), in the proximal-distal direction, in the anterior-posterior direction, or any combination of these directions. Changes in position of the femoral component can include changes along the axis of the femoral canal or changes in femoral rotation around the axis of the femoral canal, such as changes in femoral anteversion.

For any given initial position of the femoral and acetabular components, the present disclosure supports changing offsets of the various components. The acetabular offset, for example, can be changed in one direction along the vector coincident with the medial-lateral axis of the pelvis. In one or more implementations, such change can occur via a translation of the pelvis in the opposite direction of the desired change. The liner offset, for example, can be changed in three directions along the vector normal to the face of the acetabular component, which can be defined as a line connecting the center of sphere corresponding to the acetabular cup and the pole of the cup. The orientation of this vector can be dependent on the orientation of the acetabular cup (i.e., anteversion and inclination). In one or more implementations of the present disclosure, the liner offset can be changed, for example, by moving the pelvis and acetabular cup along the vector normal to the face of the acetabular component in the opposite direction of the desired change.

Moreover, the femoral offset change can represent one or more predefined changes resulting from alterations to the stem offset. For example, a change in two directions along the vector resulting from the projection of the femoral stem neck onto the plane normal to the stem's axis. Practically, this change can be implemented by moving the femur along the vector resulting from the projection of the femoral stem neck onto the plane normal to the stem's axis in the same direction as the intended change.

The femoral head offset can be a change in three directions along the vector of the trunnion of the femoral component. Note that the orientation of this vector can be dependent on the neck-shaft angle of the femoral component and the anteversion of the femoral component. Practically, this change can be implemented by moving the femur and femoral component along the vector of the trunnion of the femoral component in the same direction as the intended change.

Each of these changes to offset can be considered individually or in any simultaneous combination of two or more changes to offset. For each virtual implantation, comprising a unique configuration of implant position and implemented offsets the proposed methodology evaluates the range of motion to impingement. The evaluation of impingement can be performed at discrete functional positions of the hip, such as internal rotation during 90° flexion and neutral abduction-adduction or external rotation during 10° extension and neutral abduction-adduction. The evaluation of impingement can also be performed for the entirety of range of motion of the hip. In this scenario, impingement can be evaluated in internal rotation for all positions that involve flexion of the hip and in external rotation for all positions that involve neutral position or extension of the hip. In all cases, impingement can be defined as geometrical contact between any two components, including between femur and pelvis, femur and acetabular component, femur and liner, femoral component and pelvis, femoral component and acetabular component, and femoral component and liner. A situation can occur where impingement is not detected for the 360° of rotation of the hip along any given axis at any given hip position. In such case, the proposed algorithm can report no impingement or 360° as the range of motion to impingement in that position along that axis. Furthermore, at each position of the hip, the orientation of the pelvis relative to the femur can be adjusted to reflect, for example, patient-specific changes in pelvic tilt with hip flexion. To this end, the changes in pelvic position can be determined in at least two positions (e.g., sitting and standing) from plain radiographs or any other suitable imaging technique. The changes in pelvic position can be obtained by any other suitable measurement methods, like wearable sensors. The relationship between hip flexion and changes in pelvic position (i.e., tilt) can be obtained and formulated as an equation that can be assumed, for example, linear. Ideally, the pelvic positions are evaluated at or close to the extremes of range of motion. Then, the position of the pelvis, acetabular component, and liner are changed according to the said equation relating hip flexion with changes in pelvis orientations to account for such changes in orientation on the determined impingement. It should be noted that, while only pelvic tilt is described here, other pelvic motions, like axial rotation or lateral tilt can also be included.

The proposed methodology can be implemented as part of an optimization routine to determine the optimal combination of component choice, position, orientation, and offsets that maximizes the range of motion to impingement during functional activities.

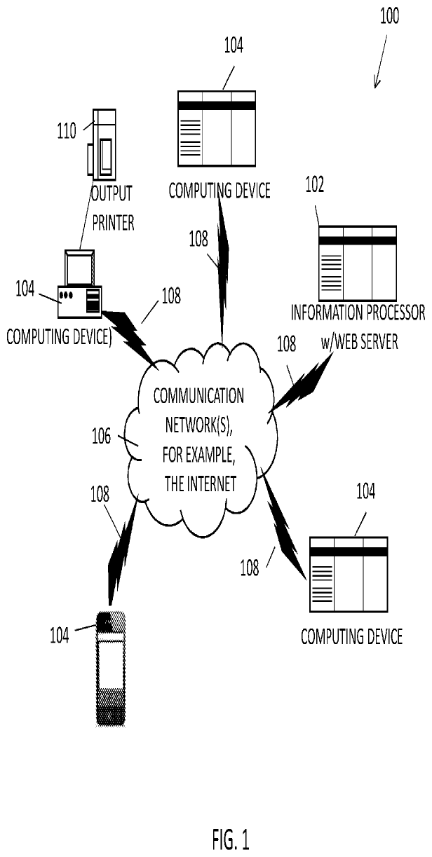

Referring now to FIG. 1, a block diagram is shown illustrating an example implementation of the present disclosure and that represents an association of a plurality of devices and the flow 108 of information associated with the devices. In the example shown in FIG. 1, various computing devices 102 and 104 are shown, each capable of executing desktop and/or mobile computing device web browser application(s) including MICROSOFT EDGE, INTERNET EXPLORER, CHROME, FIREFOX, and other (e.g., SAFARI, OPERA). In addition to standard web browser application functionality, user information can be gathered via Push Notifications, and information can be retrieved from a computing device using a “REST” interface. Various mobile devices running different operating systems are shown, including IOS, ANDROID and other (e.g., PALM, WINDOWS or other mobile device) operating system.

In the example shown in FIG. 1, one or more data processing apparatuses 102 can be operatively coupled to one or more user computing device(s) 104. Devices 102/104 can be respectively operated by one or more users skilled in the use of the proposed workflow, included, but not limited to, healthcare providers and associated staff, medical specialists, and/or biomechanical specialists. Healthcare providers can include, for example, physicians, physician assistants, nurses, therapists and/or other providers of healthcare services. Biomechanical specialist can include, for example, engineers specialized in biomechanics. Data processing apparatus 102 and/or user computing device 104 can be operable to access and/or store various information on database(s) including, for example, historic medical and procedure information patients, physicians, devices, or the like.

Also illustrated in FIG. 1 is a network 106, which can be configured as a local area network (LAN), wide area network (WAN), Peer-to-Peer network (“P2P”), Multi-Peer network, the Internet, one or more telephony networks or a combination thereof, that is operable to connect data processing apparatus 102 and/or devices. Though many of the examples and implementations shown and described herein relate to product and/or service recommendations, many other forms of content can be provided and/or delivered by system 100.

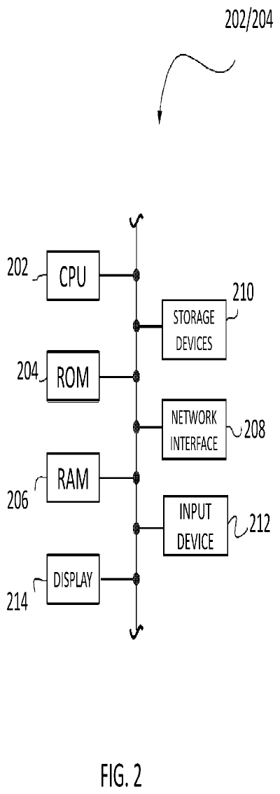

FIG. 2 is a block diagram that illustrates functional elements of one or more of data processing apparatus 102 or computing device 104 and preferably include one or more central processing units (CPU) 202 used to execute software code in order to control operations, including of data processing apparatus 102, read only memory (ROM) 204, random access memory (RAM) 206, one or more network interfaces 208 to transmit and receive data to and from other computing devices across a communication network, storage devices 210 such as a hard disk drive, solid state drive, floppy disk drive, tape drive, CD-ROM or DVD drive for storing program code, databases and application code, one or more input devices 212 such as a keyboard, mouse, track ball and the like, and a display 214.

The various components of devices 102 and/or 104 need not be physically contained within the same chassis or even located in a single location. For example, storage device 210 can be located at a site which is remote from the remaining elements of computing devices 102 and/or 104 and can even be connected to CPU 202 across communication network 106 via network interface 208.

The functional elements shown in FIG. 2 (designated by reference numbers 202-214) are preferably of the same categories of functional elements preferably present in computing device 102 and/or 104. However, not all elements need be present, for example, storage devices in the case of mobile computing devices (e.g., smartphones), and the capacities of the various elements are arranged to accommodate expected user demand. For example, CPU 202 in computing device 104 can be of a smaller capacity than CPU 202 as present in data processing apparatus 102. Similarly, it is likely that data processing apparatus 102 will include storage devices 210 of a much higher capacity than storage devices 210 present in computing device 104. Of course, one of ordinary skill in the art will understand that the capacities of the functional elements can be adjusted as needed. For example, one or more graphics processing units (GPU) can be utilized for processing and providing functionality shown and described herein. In addition, or in the alternative, a cluster of computing devices can work to provide functionality shown and described herein.

The nature of the present disclosure is such that one skilled in the art of writing computer executed code (software) can implement the described functions using one or more or a combination of a popular computer programming language including but not limited to C++, JAVA, ACTIVEX, HTML, XML, ASP, SOAP, IOS, OBJECTIVE C, ANDROID, TORR, PYTHON, MATLAB, and various web application development environments.

As used herein, references to displaying data on computing device 104 refer to the process of communicating data to the computing device 104 across communication network 106 and processing the data such that the data can be viewed on the user computing device 104 display 214 using a web browser, custom application or the like. The display screens on computing devices 102/104 present areas within system 100 such that a user can proceed from area to area within the system 100 by selecting a desired link. Therefore, each user's experience with system 100 will be based on the order with which (s)he progresses through the display screens. In other words, because the system is not completely hierarchical in its arrangement of display screens, users can proceed from area to area without the need to “backtrack” through a series of display screens. For that reason and unless stated otherwise, the following discussion is not intended to represent any sequential operation steps, but rather the discussion of the components of system 100.

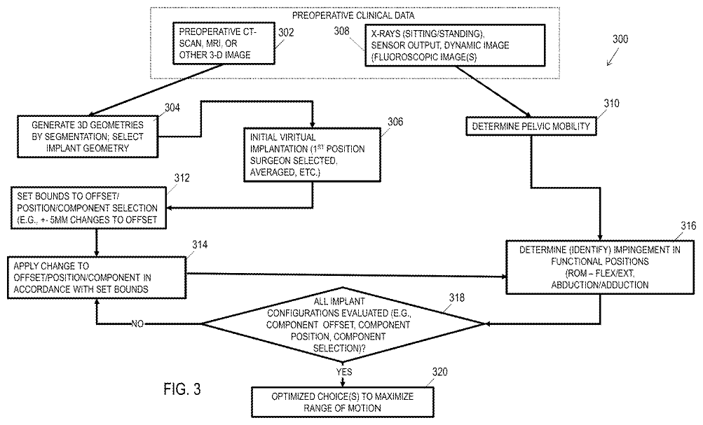

FIG. 3 illustrates a computational workflow 300, in accordance with an example implementation of the present disclosure. It is to be appreciated that several of the logical operations described herein can be implemented as a sequence of computer-implemented acts or program modules running on one or more computing devices. In one or more implementations, particular software can be used for various tasks, such as like segmentation software (e.g., MIMICS) to obtain bony geometries, CAD software (e.g., GEOMAGIC DESIGN X) to reproduce the virtual implantation, and simulation software (e.g., ADAMS or ABAQUS) to determine impingement.

Accordingly, the operations described herein, including logical operations, are referred to variously as operations, steps, structural devices, acts and modules can be implemented in software, in firmware, in special purpose digital logic, and any combination thereof. It should also be appreciated that more or fewer operations can be performed than shown in the figures and described herein. These operations can also be performed in a different order than those described herein.

In accordance with the algorithmic steps in the example high-level process shown in FIG. 3, anatomical information about a patient in the form of images or data, such as output from one or more sensor devices, is provided to one or more computing devices for processing. For example, at step 302, one or more preoperative three-dimensional images, such as CT scans, MRIs, or other three-dimensional image is provided to an information processor 102 or computing device 104. At step 304, 3D geometries of the bones, including pelvis and femur, are generated by segmentation, as known in the art, and implant geometry associated with one or more respective implants is selected. For example, a respective implant component identifier is looked-up in one or more databases, and information associated with the geometry of the respective implant component is matched with the identifier.

At step 306, using the bony geometries and selected implant geometry at step 304, an initial determination can be made to provide a virtual implantation. For example, the initial determination can be based on pre-set defaults, or a surgeon selection made in a graphical user interface, or generated automatically based on the patient's anatomy or other suitable technique.

In addition, at step 308 images such as x-rays, fluoroscopic images, or data, such as output from one or more sensors is provided to an information processor 102 or computing device 104. Thereafter, using the information received in step 308 and, thereafter, pelvic mobility is determined (step 310). This information is utilized when determining the range of motion to impingement, in step 316, to account for the patient-specific pelvic mobility at each functional position.

Continuing with reference to the example process shown in FIG. 3, at step 312 bounds are set, such as in connection with offset, position, and/or component selection. For example, ±5 mm changes to offset can be defined at step 312 as a function of information received in a graphical user interface, or other suitable technique, such as automatically retrieved or calculated based on the characteristics of the patient and the implant. Thereafter, a looping process is used including at step 314 in which a change to the offset, position, or component selection is applied in accordance with the set bounds. Thereafter, the process flows to step 316 and impingement is determined or identified in respective functional positions, such as in connection with range of motion (e.g., flexion/extension, abduction/adduction). At step 318, a determination is made whether all implant configurations (e.g., component offset, component position, and component selection) have been evaluated. If the result of the determination at step 318 is no, then the process branches back to step 314 and another change to offset, position, or component selection is applied. Alternatively, if the determination at step 318 is yes, then the process branches to step 320, and the choice of implant design, position, orientation, and offset to maximize range of motion is identified and output. For example, a notification can be provided, such as an audible alert or a display in a graphical user interface. Thereafter, the process ends (not shown). It should be noted that the implant configuration (design, position, orientation, and offset) that maximizes offset for a given functional position (e.g., 90° flexion) may be different than the configuration that maximizes offset in a different motion (e.g., 10° extension). As such, the process may determine a plurality of configurations that maximize range of motion to impingement under each different motion.

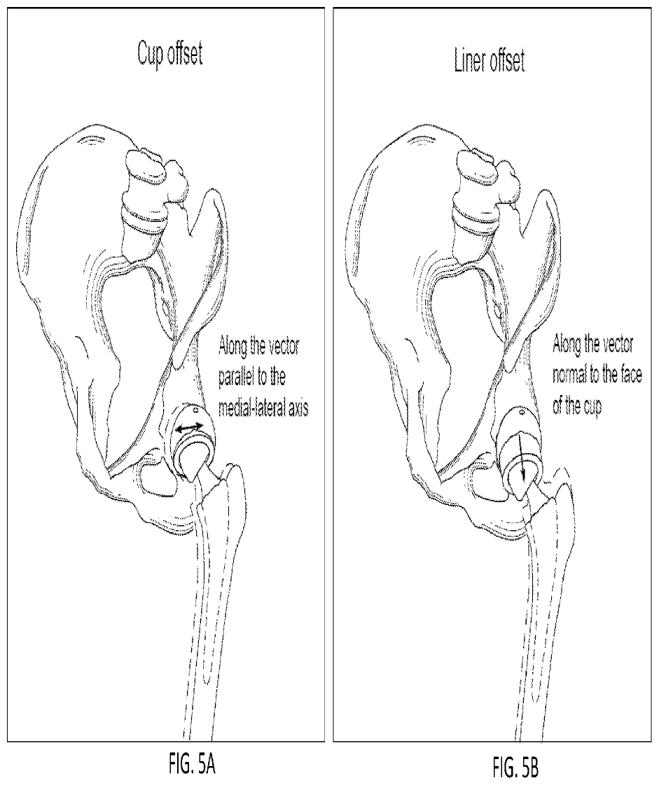

FIGS. 5A and 5B are anatomical renderings illustrating acetabular cup offset and liner offset, respectively. As shown in FIGS. 5A and 5B, the femur and/or implant change from their greyed out initial position to their final position in a direction, which can depend upon the component on which the offset is considered. For example, offsets to the cup result in a translation along a vector parallel to the medial-lateral axis of the pelvis. Offsets to the liner result in a translation along a vector perpendicular to the face of the acetabular cup.

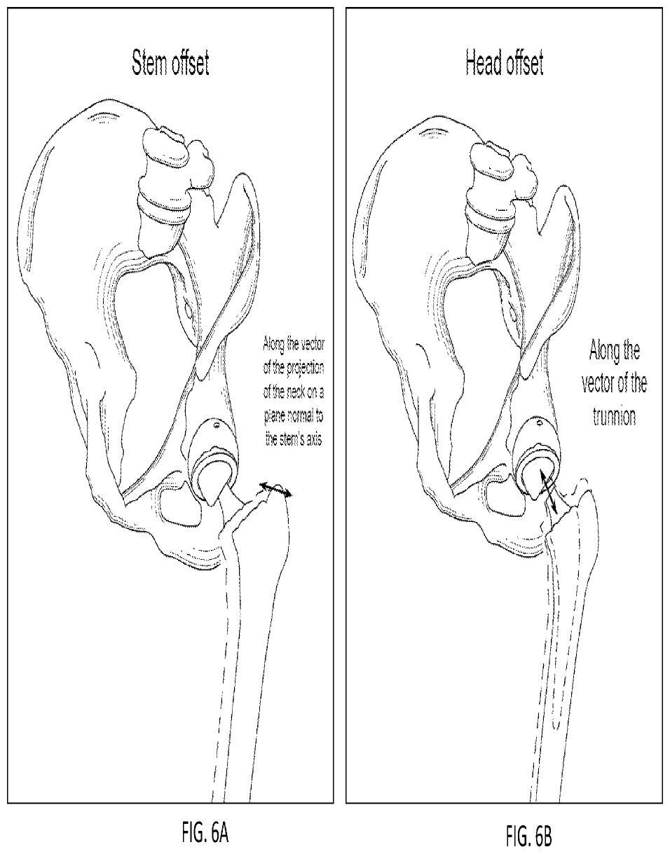

FIGS. 6A and 6B are anatomical renderings illustrating femoral stem offset and femoral head offset, respectively. As shown in FIGS. 6A and 6B, offsets to the femoral stem result in a translation along the vector resulting from the projection of the femoral neck into a plane perpendicular to the stem's axis. Offsets to the femoral head result in a translation along the vector of the trunnion of the femoral implant.

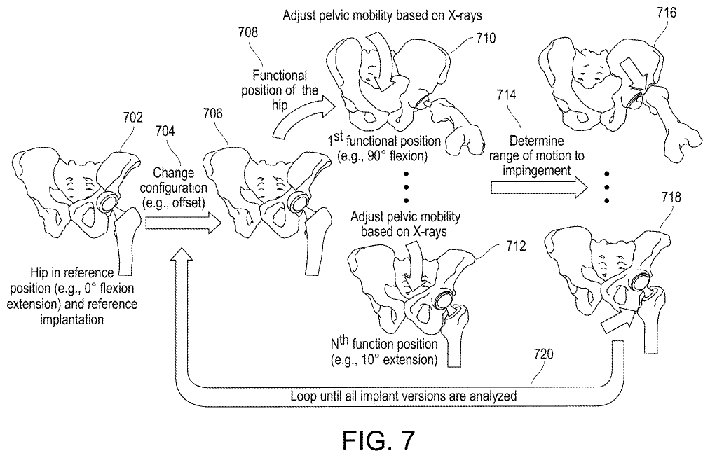

FIG. 7 graphically illustrates another computational workflow, in accordance with an example implementation of the present disclosure involving computer modeling. The process starts with the hip in a reference position (e.g., 0° of flexion/extension) and with the implants in their reference starting position. The implant configuration (e.g., implant position, orientation, design, or offset) is changed in step 704, according to pre-set boundaries to generate a new configuration, 706. The hip is then placed in a plurality of functional positions, in step 708, where the pelvic orientation is adjusted based on patient-specific data, as described previously. For example, a first position 710 can involve hip flexion and a “nth” functional position, 712, can involve hip extension. The range of motion to impingement is determined (step 714). Impingement for each position can occur during internal rotation, 716 or external rotation, 718. Impingement can be displayed graphically (steps 716, 718) for any of the configurations evaluated. The process involving changing the configuration (704), placing the hip in functional positions (708), and determining impingement (714) is performed in a loop 720, until all predefined implant configurations have been evaluated. Once all implant configurations have been evaluated, the process produces at least one configuration that maximizes range of motion to impingement in at least one functional position.

Although the present disclosure is described by way of example herein in terms of a web-based system using web browsers, custom applications and a web site server (data processing apparatus 102), and with mobile computing devices, system 100 is not limited to that particular configuration. It is contemplated that system 100 can be arranged such that computing device 104 can communicate with, and display data received from, data processing apparatus 102 using any known communication and display method, for example, using a non-Internet browser Windows viewer coupled with a local area network protocol such as the Internetwork Packet Exchange (IPX). It is further contemplated that any suitable operating system can be used on computing device 104, for example, WINDOWS, MAC OS, OSX, LINUX, IOS, ANDROID and any suitable PDA or other computer operating system.

As used herein, the terms “function” or “module” refer to hardware, firmware, or software in combination with hardware and/or firmware for implementing features described herein. In the hardware sense, a module can be a functional hardware unit designed for use with other components or modules. For example, a module may be implemented using discrete electronic components, or it can form a portion of an entire electronic circuit such as an Application Specific Integrated Circuit (ASIC). Numerous other possibilities exist, and those of ordinary skill in the art will appreciate that the system can also be implemented as a combination of hardware and software modules. In the software sense, a module may be implemented as logic executing in a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, Perl, or Python. It will be appreciated that software modules may be callable from other modules or from themselves, and/or may be invoked in response to detected events or interrupts. Software instructions may be embedded in firmware. Moreover, the modules described herein can be implemented as software modules, but may be represented in hardware or firmware. Generally, the modules described herein refer to logical modules that may be combined with other modules or divided into sub-modules despite their physical organization or storage.

Thus, as shown and described herein, the present disclosure provides a computational framework capable of predicting impingement and, accordingly, risk of dislocation. More particularly, increasing offset by any method has been found to reduce impingement. Center-of-rotation offset changes via acetabular cup or liner have been shown to have the greatest impact on extra-prosthetic impingement.

As noted herein, predicting impingement in THA is significant as being a likely precursor of dislocation, as well as the most common complication after THA. The present disclosure includes systems and methods to quantify the effect of THA component offset changes on the range of motion to bone-bone impingement. As shown and described herein, preoperative images and data can be processed to create 3D computational models with realistic implantations, which are usable to evaluate range of motion to impingement. Variables, such as changes to cup offset, liner offset, head offset, and stem offset are factored, as well as biplanar radiographs of patients sitting and standing to quantify and incorporate the pelvic mobility in the models.

Moreover, the present disclosure can be used to compare changes that affect the hip center of rotation (i.e., changes to acetabular offset through cup or liner offset) to changes that affect the position of the femur without affecting the center of rotation (i.e., changes to femoral offset through head or stem offset) as it relates to their relative impact in the range of motion to bone-bone impingement. While changes to acetabular and femoral offset may have similar directions when analyzed in static AP plain radiographs, the present disclosure allows for taking into account the three-dimensional nature of these changes, particularly during the functional positions associated with impingement such flexion and internal rotation or extension and external rotation. This underscores a technological improvement provided by the present disclosure underscoring the importance of functional component planning in impingement-prone positions. Surgical choices that lateralize the femur may not change the rotation path of the femur thereby impacting the range of motion only if the bone-bone anatomy changes at the impingement location. Conversely, surgical choices that involve the acetabular component can result in a combined lateralization of the femur, with respect to the pelvis and a change in the center of rotation. Such change in center of rotation can affect the arc of motion path as the femur rotates, leading to a greater effect in the range of motion than femoral offset changes alone, irrespective of the bone-bone anatomy at the impingement location. These effects of multidimensional changes to offset and the effect of altering the center of rotation require advanced analysis techniques. The present disclosure provides a framework for taking into account these changes in a subject-specific manner to provide a recommendation of the implant configuration that maximizes the range of motion to impingement. From a clinical standpoint, surgeons considering adding offset to a total hip arthroplasty construct in the operating room to reduce impingement can now, in view of the teachings herein, perform an informed decision on prioritizing the methods of increasing offset based on which method provides the greatest benefit to the range of motion for any particular patient under functional motions. Options made possible in accordance with the present disclosure can be particularly useful to identify anterior impingement, which can involve bone-bone extra-prosthetic impingement of the greater trochanter or femoral neck on the AIIS or ilium. The present disclosure can be particularly useful also to identify posterior impingement, which can involve bone-bone extra-prosthetic impingement of the lesser trochanter on the ischium.

Moreover, the present disclosure identifies, can be utilized to determine the specific effect of each implant configuration on the range of motion at each functional position. For example, the implant configuration that provides the greater increase in range of motion in flexion and internal rotation may differ from the configuration that maximizes range of motion in extension and external rotation. For example, in flexion, the liner offset can be more advantageous than the cup offset in increasing range of motion to impingement due to the more anterior position of the center of rotation and the femur. Conversely, the anteversion of the cup can decrease the effect of acetabular liner offset for reducing impingement in extension. The present disclosure can identify a plurality of configurations and provide, for example, a solution that maximizes the overall range of motion, or a solution that favors increasing the range of motion in a predetermined functional position, or alternatively can present all solutions to the surgeon, who can then decide the best configuration for the specific patient.

While operations shown and described herein may be in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the embodiments described above should not be understood as requiring such separation in all embodiments, and it should be understood that the described program components and systems can generally be integrated together in a single software product or packaged into multiple software products.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this disclosure, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particular embodiments of the subject matter described in this disclosure have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing can be advantageous.

What is claimed:

1. A computer-implemented method for virtually optimizing at least one aspect of an implantation to maximize range of motion in connection with total hip arthroplasty, the method comprising:
receiving, by at least one computing device configured by executing instructions stored on non-transitory processor readable media, preoperative clinical data of a patient;
generating, by the at least one computing device, three-dimensional geometries of the patient's bones using the preoperative clinical data;
selecting, by the at least one computing device, implant geometry associated with at least one implant;
determining, by the at least one computing device using the geometries of the bones and the selected at least one implant, a virtual implantation including a plurality of components;
defining, by the at least one computing device, bounds in connection with the virtual implantation, including for at least one of component selection, position, orientation, and offset;
applying, by the at least one computing device, a change to an aspect of the virtual implantation in accordance with the bounds;
determining, by the at least one computing device using at least some of the preoperative clinical data, pelvic mobility;
determining, by the at least one computing device using the applied change to the aspect of the virtual implantation and the determined pelvic mobility, impingement resulting from at least one position of the patient's hip;
evaluating, by the at least one computing device after determining the impingement, at least one choice associated with the at least one implant configuration; and
where the step of evaluating does not represent the choices are optimized:
applying, by the at least one computing device, a different change to an aspect of the virtual implantation, in accordance with the bounds;
determining, by the at least one computing device, impingement resulting from at least one position of the patient's hip; and
evaluating, by the at least one computing device after determining the impingement, at least one choice associated with an aspect of the virtual implantation;
where the step of evaluating does represent the choices are optimized:
outputting, by the at least one computing device, information representing maximized offset.

2. The method of claim 1, wherein the preoperative data includes at least one of a computed tomography scan, a magnetic resonance image, an x-ray, dynamic image output, and sensor output.

3. The method of claim 1, wherein the three-dimensional geometries of the bones are generated by segmentation.

4. The method of claim 1, wherein selecting the implant geometry includes:
identifying, by the at least one computing device, a component identifier; and
matching, by the at least one computing device, information stored in at least one database representing the implant geometry.

5. The method of claim 1, wherein the virtual implantation is determined using at least one of an anatomical-based position according to established clinical protocols and a user selection.

6. The method of claim 1, wherein defining the bounds is based on at least one of received information, information retrieved from a database, and information calculated using characteristics of a patient and an implant.

7. The method of claim 1, wherein the at least one functional position includes at least one of flexion, extension, abduction, adduction, internal rotation, and external rotation.

8. The method of claim 1, the virtual implantation includes at least one of component selection, component position, component orientation, and component offset.

9. The method of claim 8, wherein the choices include component selection, position, orientation, and offset.

10. A computer-implemented system for virtually optimizing at least one aspect of an implantation to maximize range of motion in connection with total hip arthroplasty, the system comprising:
at least one computing device configured by executing instructions stored on non-transitory processor readable media, including to perform steps including:
receiving preoperative clinical data of a patient;
generating three-dimensional geometries of the patient's bones using the preoperative clinical data;
selecting implant geometry associated with at least one implant;
determining, using the geometries of the bones and the selected at least one implant, a virtual implantation including a plurality of components;
defining bounds in connection with the virtual implantation, including for at least one of component selection, position, orientation, and offset;
applying a change to an aspect of the virtual implantation in accordance with the bounds;
determining, using at least some of the preoperative clinical data, pelvic mobility;
determining, using the applied change to the aspect of the virtual implantation and the determined pelvic mobility, impingement resulting from at least one position of the patient's hip;
evaluating, after determining the impingement, at least one choice associated with the at least one implant configuration; and
where the step of evaluating does not represent the choices are optimized:
applying a different change to an aspect of the virtual implantation, in accordance with the bounds;
determining impingement resulting from at least one position of the patient's hip; and
evaluating, after determining the impingement, at least one choice associated with an aspect of the virtual implantation;
where the step of evaluating does represent the choices are optimized:
outputting information representing maximized offset.

11. The system of claim 10, wherein the preoperative data includes at least one of a computed tomography scan, a magnetic resonance image, an x-ray, dynamic image output, and sensor output.

12. The system of claim 10, wherein the three-dimensional geometries of the bones are generated by segmentation.

13. The system of claim 10, wherein selecting the implant geometry includes:
identifying a component identifier; and
matching information stored in at least one database representing the implant geometry.

14. The system of claim 10, wherein the virtual implantation is determined using at least one of an anatomical-based position according to established clinical protocols and a user selection.

15. The system of claim 10, wherein defining the bounds is based on at least one of received information, information retrieved from a database, and information calculated using characteristics of a patient and an implant.

16. The system of claim 10, wherein the at least one functional position includes at least one of flexion, extension, abduction, adduction, internal rotation, and external rotation.

17. The system of claim 10, the virtual implantation includes at least one of component selection, component position, component orientation, and component offset.

18. The system of claim 17, wherein the choices include component selection, position, orientation, and offset.
